(19) United States
(12) Patent Application Publication
(10) Pub. No.: US 2005/0191346 A1
(43) Pub. Date: Sep. 1, 2005

(54) Delivery capsules

(76) Inventors: Edward Nowak, Cambridge (GB); Barry John Muncaster, Cambridge (GB); Malcolm David Brown, Norfolk (GB)

Correspondence Address: MORGAN & FINNEGAN, L.L.P., 3 WORLD FINANCIAL CENTER, NEW YORK, NY, 10281-2101, US

(21) Appl. No.: 10/701,293

(22) Filed: Nov. 3, 2003

Related U.S. Application Data
(63) Continuation of application No. 10/030,902, filed on Jan. 9, 2002, now abandoned.

(30) Foreign Application Priority Data
Jul. 9, 1999 (GB) 99160335

(51) Int. Cl. A61K 9/48

(52) U.S. Cl. 424/451

(57) ABSTRACT

A delivery capsule, designed to retain and protect its contents until an intended site of delivery or conditions of delivery are encountered, has at least two separate chambers (18, 20), the chambers usually containing different materials. The capsule is preferably internally divided by a dividing wall or septum (16), conveniently in the form of a median wall symmetrically arranged to form two chambers of similar size and shape. Also disclosed are a method of encapsulation and encapsulation apparatus.

### FIELD OF THE INVENTION

This invention relates to a delivery capsule, that is, a capsule designed to retain and protect its contents until an intended site of delivery or conditions of delivery are encountered, at which point the capsule contents are released.

### BACKGROUND TO THE INVENTION

Delivery capsules are well known and find particular application in the form of ingestible gelatin capsules for the delivery of accurately metered doses of pharmaceutical preparations and dietary supplements. Liquid preparations are typically encapsulated in soft gelatin capsules and particulate or powdered preparations are typically encapsulated in two part hard gelatin capsules. The capsules are designed to release their contents after ingestion, typically by solution of the capsule wall, and by use of suitable capsule material can thus provide a means of administering a dose of a preparation at a desired appropriate site in the body. The finished capsules offer protection to the contents yet solubility within the body.

Other uses of delivery capsules include delivery of cosmetic ingredients, eg fragranced bath oils encapsulated in soft gelatin capsules for release into bath water, paint balls in the form of paint-containing capsules that rupture on impact etc.

There are limitations on current capsules and encapsulation techniques. For example, because of differences in powder and liquid handling, the processing means for the encapsulation of powders and liquids within a gelatin capsule are quite distinct and incompatible. This situation renders impossible the provision of a gelatin capsule containing both powder and liquid that are kept separate.

The present invention seeks to address certain shortcomings and limitations of current capsules and encapsulation techniques.

### SUMMARY OF THE INVENTION

In one aspect the invention provides a delivery capsule having at least two separate chambers.

The chambers of the capsule are completely discrete and separated from each other so that no communication between the chambers is possible. This means that the contents of the different chambers are kept separate from each other within the capsule until delivery.

In most cases, different chambers of the capsule will contain different materials, possibly in different physical forms, eg liquid, solid (eg tablet, particulate, powdered), slurry etc, in a way that has not hitherto been possible, although it is also possible for the different chambers to contain separate doses of the same material.

The capsule is preferably internally divided by a dividing wall or septum, conveniently in the form of a median wall symmetrically arranged to form two chambers of similar size and shape.

One or more chambers of the capsule may be further divided if required, eg by inclusion in a chamber of a smaller delivery capsule, constituting a further separate chamber.

The invention thus provides a compartmented capsule in a way that has not been done hitherto. Indeed, it is believed that this is not possible with known techniques for gelatin encapsulation.

Instead of using gelatin for encapsulation, the present invention preferably uses a heat-sealable material that is capable of deforming plastically on heating (a thermoplastic material) and/or that is capable of deforming plastically when partially solvated by application of an appropriate solvent. Suitable materials include hydroxy propyl methyl cellulose (HPMC), pectin, polyethylene oxide, polyvinyl alcohol, alginate, polycaprolactone, gelatinised starch-based materials etc. The material may be coated, eg with gum arabic, pectin, alginate eg sodium alginate etc to modify properties. For example, gum arabic, pectin and alginate all have a slight retarding effect on HPMC solubility, the extent of the effect varying according to coating thickness. Further, both pectin and alginate can be cross-linked, eg with calcium, this has the effect of making the material pH sensitive such that it will not dissolve in the mouth but will dissolve in the stomach where pH is lower. Multi-layer materials may also be used. Examples of suitable capsule materials and coatings are given in WO 97/35537 and WO 00/27367. The capsule materials also have the advantage compared with gelatin of being non-animal derived, and so having no possibility of transmitting animal-related diseases such as bovine spongiform encepehalopathy (BSE). Such materials are commercially available, eg in the form of ribbon-like films or can be readily manufactured, eg by extrusion from solution. One currently favoured material is the thermoplastic material HPMC, in expanded or non-expanded form, with or without coatings. HPMC is suitable for ingestion by humans and so can be used for ingestible capsules as well as other uses, eg culinary, cosmetic etc.

A compartmented capsule in accordance with the invention can be used simply to keep separate in the respective chambers two materials prior to delivery. This can be of advantage, for example, when delivering to the same site two materials which react together on admixture: by use of a compartmented capsule in accordance with the invention the two materials can be kept separate until the septum wall is dissolved on delivery, bringing the materials together. This approach is also useful, say, for delivery of two separate pharmaceutical preparations. For instance, this approach is relevant to delivery of certain multi-component cold remedies which are currently unable to get FDA approval due to concerns of possible chemical reactions prior to ingestion: by using a capsule in accordance with the invention to keep the components separate within the capsule prior to delivery such difficulties can be overcome. As a further example, there is a drug called Acctuane which is an effective treatment for acne but which can also cause birth defects. In order to ensure that this does not occur birth control drugs should be taken simultaneously with Acctuane by fertile female users. For safety reasons it would thus be far preferable if the birth control drug and the acne remedy were taken together, but kept separate until after ingestion. This can be readily achieved by use of a compartmented capsule in accordance with the invention.

Furthermore, by using different materials (either in terms of thickness and/or composition and/or coatings) defining the different chambers of the capsule, it is possible to arrange for release of the contents of the different chambers under different conditions, eg at different specific sites within the body. The contents of different compartments can thus be targeted to different specific areas within the body.

For instance, use of a thicker material defining one compartment may result in slightly delayed release of material compared with that from a compartment defined by a thinner layer of similar material.

Another example is the use of a pH sensitive coating on the material defining one chamber so that chamber contents are released at different delivery sites dependent upon pH. Use of enteric coatings such as cellulose acetate phthalate can also be used to target release, eg to within the stomach. Coatings such as ethyl cellulose can be used to retard solubility times. A further example is use of expanded HPMC defining one compartment and non-expanded HPMC defining another compartment. Expanded HPMC film releases rapidly in the mouth while standard, non-expanded film has sufficient resistance to dissolution to release only after it has been swallowed, providing that it is not kept in the mouth too long.

It is also possible to coat a finished capsule after formation with materials such as sodium alginate to improve robustness or alter solubility.

The capsule materials may include optional colourings, eg in the form of known food dyes such as FD and C yellow number 5, optional flavourings, textures etc.

The capsules may have a range of different sizes and shapes as appropriate dependent on intended usage. Capsules are typically generally spherical, ovoid, cylindrical etc in shape, preferably incorporating a median septum as described above. Typical maximum dimensions of the capsule are in the range 3 mm to 20 mm, but other sizes are possible.

The capsules are conveniently made by a vacuum or pressure forming technique, that may be loosely based on the technique described in WO 97/35537 but with very substantial modification.

In a further aspect, the invention thus provides a method of encapsulation, comprising supplying two films of material capable of deforming plastically on heating and/or when partially solvated; heating the films and/or applying solvent; forming the films into suitably shaped capsule portions; supplying respective substances to be encapsulated to capsule portions of each film; supplying a film of a dividing septum material to at least one of the filled capsule portions; sealing the capsule portions and septum material together to form a capsule having at least two separate chambers.

The films are preferably formed into capsule portions by application of elevated pressure or vacuum (or reduced pressure).

It is preferred to use two layers of film for producing the septum, with one film applied to each respective capsule portion, as handling including optional coating is easier.

Adhesive material is preferably applied to the various film materials to help secure the capsule portions and septum together. Capsule sealing is preferably accomplished by heat sealing, to fuse the films of material together, although other sealing methods may be used.

Pre-formed films of material may be used. Alternatively, the films may be formed during the encapsulating process, eg by being cast from solution.

In a further aspect the invention provides encapsulation apparatus, comprising means for supplying two films of material to an encapsulation unit; means for plastically deforming each film to form suitably shaped capsule portions; means for supplying respective substances to be encapsulated to the respective capsule portions of each film; means for supplying a film of dividing septum material to at least one of the filled capsule portions; and means for sealing together the capsule portions and septum material to produce a capsule having at least two separate chambers.

The apparatus typically also comprises reservoirs of the substances to be encapsulated, with associated supply arrangements adapted to supply a metered doses of the substance to the capsule portions at predetermined time intervals. The arrangement may employ syringe pumps or the like.

The apparatus conveniently includes heater means for heating the capsule film material to enable thermoplastic deformation.

The means for deforming the films conveniently comprises a pair of similar vacuum belts.

The invention is applicable to encapsulation of a wide range of pharmaceutical, culinary, cosmetic etc ingredients, enabling delivery to different sites of different materials or delivery to the same site of materials that are desirably kept separate prior to delivery.

Capsules described in this specification provide a delivery means with either at least two distinct liquid or solid, eg powder fills, or a combination of liquid and solids, eg powder. The materials can also be selected so as to exclude gelatin. The combination of materials used for the capsule wall and capsule dividing septum can be chosen to release either or both parts of the contents of capsule at specific sites within the body. These components can then address two different specific areas or act synergistically when mixed on release at the same site. In the latter example the capsule is serving to prevent the mixing of the two components prior to them reaching the correct site within the body as well as providing an accurate dose and blend of components for maximum efficacy.

The present invention enables the encapsulation of both powders and liquids within discrete chambers in an ingestible capsule. Using pre-formed rolls of film such as hydroxy propyl methyl cellulose capsules are formed with an outer shell and a dividing septum. In such a capsule two different materials which would react if brought together in a single chamber can be kept apart until the septum wall is dissolved.

By the application of surface coatings to the forming rolls and the dividing layer prior or post capsule formation, or the use of different materials for the forming rolls, capsules can be formed which release their contents under different environmental conditions. An example of this is the application to pH sensitive coatings on the outer surface of the capsule wall and septum which causes the two distinct chambers to release their contents at different delivery sites dependent upon the pH of the surrounds.

The capsules are produced on dedicated machinery employing the use of vacuum forming and heat sealing, and can be filled with liquids or powders.

The invention will be further described, by way of illustration, with reference to the accompanying drawings in which:

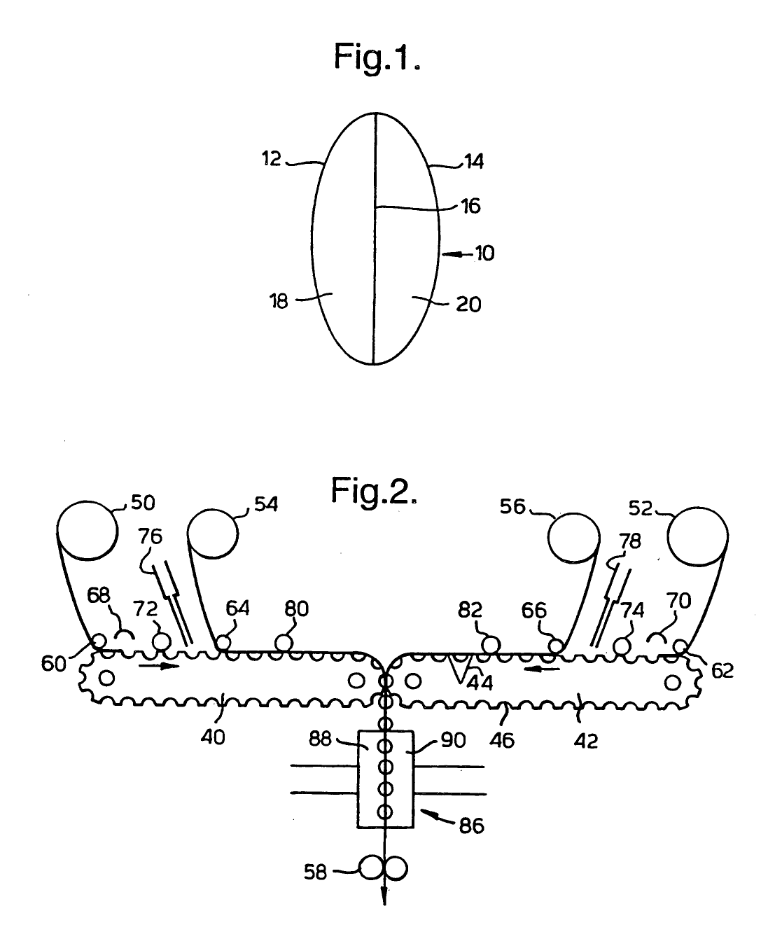

FIG. 1 is a schematic sectional view of a delivery capsule in accordance with the invention; and

FIG. 2 is a schematic representation of one embodiment of apparatus in accordance with the invention for producing a delivery capsule embodying the invention.

### DETAILED DESCRIPTION OF THE DRAWINGS

Referring to the drawings, FIG. 1 illustrates schematically a generally ovoid delivery capsule 10 comprising at outer shell or wall in the form of two similar half shells 12 and 14 each of generally semi-ovoid form, and a median dividing wall of septum 16 that divides the capsule into two similar chambers or compartments 18 and 20 that are completely separate from each other, with no communication between the chambers 18 and 20 being possible.

Each chamber 18 and 20 contains a metered amount of a different material (not shown), eg with a powdered or particulate material in chamber 18 and a liquid material in chamber 20, or visa versa, or with different liquid materials in each of the two chambers or with different powdered or particulate materials in each of the two chambers.

The half shells 12 and 14 of the septum 16 may be made of similar or different materials, depending on the desired properties and intended use of the capsule.

For example, where the function of the compartments is simply to keep two materials separate from each other until release at the same site of delivery, thus can be achieved by all of the capsule walls, half shells 12 and 14 and septum 16, being of the same material, eg HPMC (possibly coated as discussed above).

However, where the capsule is designed to delivery the contents of chamber 18 and chamber 20 at different sites or under different conditions, eg at different sites in the body after ingestion, it is appropriate for the capsule walls to be of different material, eg with half shell 12 of a first material and half shell 14 and septum 16 of a second, different material, with the two different materials functioning to release the contents of the associated compartment under different conditions, eg under different conditions of pH, or after different time intervals etc. For example, the first material may comprise pectin and the second material may comprise HPMC. As a further example, the first material may comprise un-coated HPMC and the second material may comprise a HPMC coated, eg with sodium alginate. Another possibility is for the first and second materials to have different coatings, eg of sodium alginate and gum arabic. A yet further possibility is for the first material to be expanded HPMC, with the second material being standard cast HPMC coated with sodium alginate.

It is also possible for septum 16 to be of completely insoluble material that will, eg, pass through the body unchanged.

The dimensions of capsule 10 may be varied to suit the intended purpose of the capsule, with the maximum dimension typically being in the range 3 mm to 20 mm.

### EXAMPLES

The following examples serve to give specific illustrations of this invention but they are not in any way intended to limit the scope of this invention.

### Example 1

A dual delivery capsule as shown in FIG. 1 where the septum 16, and the capsule walls 12 and 14 are of like material, exampled by hydroxy propyl methyl cellulose.

### Example 2

A dual delivery capsule as shown in FIG. 1 with one wall and dividing septum of like material, exampled by hydroxy propyl methyl cellulose, and the other wall of different material, exampled by pectin.

### Example 3

A dual delivery capsule as shown in FIG. 1 with walls and dividing septum of like material, exampled by hydroxy propyl methyl cellulose with a coating on one half of the capsule and one side of the capsule dividing septum, exampled by sodium alginate.

### Example 4

A dual delivery capsule as shown in FIG. 1 with walls and dividing septum of like material, exampled by hydroxy propyl methyl cellulose with the same coating on both sides of the capsule, exampled by sodium alginate.

### Example 5

A dual delivery capsule as shown in FIG. 1 with walls 12 and 14 of like material exampled by hydroxy propyl methyl cellulose with different coatings on each exampled by sodium alginate and gum arabic and dividing septum 16 coated on the side closest to the wall bearing the alginate coating with gum arabic.

### Example 6

A dual delivery capsule as shown in FIG. 1 with a liquid fill contained in chamber 20 exampled by dextromethorphan and a powder filled example by chlopheniramine contained in chamber 18 between septum 16 and capsule wall 12.

### Example 7

A dual delivery capsule as shown in FIG. 1 with two different liquid fills exampled by cod liver oil and evening primrose oil contained in chamber 20 and chamber 18, respectively.

FIG. 2 illustrates schematically one embodiment of apparatus for producing capsules in accordance with the invention.

The illustrated encapsulation apparatus comprises two similar, aligned, side-by-side vacuum belts 40 and 42 each comprising a plurality of articulated segments of plastics-coated aluminium as represented by segment 44. Each segment has a width of about 600 mm, extending perpendicular to the plane of the sectional view of FIG. 2, and is formed with a row of hemi-ovoid recesses running across its width, eg recess 46, only one such recess of each segment being visible in the drawing. Drive means (not shown) are provided for driving the two belts synchronously, with belt 40 being driven in a clockwise direction and belt 42 being driven in an anticlockwise direction, with the recesses of the two belts in registration with each other. Each recess includes a number of fine bore vacuum ports (not shown), each about 0.4 mm in diameter, with vacuum means (not shown) arranged to apply a vacuum in the range −15 to −30 inches mercury. The vacuum may be applied only to the recesses in the segments when in the upper portion of travel of the belts.

Four rolls of film material 50, 52, 54 and 56 are rotatably supported on respective spindles, with the films being pulled from the spindles and over vacuum belts by a driven nip roller 58. The films pass around respective guide rollers 60, 62, 64, 66 to be brought into contact with the associated vacuum belt.

Films 50 and 52 form the generally hemi-ovoid outer shell halves of a capsule. To this end, the films pass below respective infra red heaters 68 and 70 located near the outer end of each vacuum bed, which act to heat the film passing there below to a temperature at which it is capable of deforming plastically. The films then deform to take up the shape of the recesses in the vacuum belts, assisted by the vacuum applied to the belts.

The films, moving with the vacuum bed, then pass below respective adhesive application stations 72, 74 in the form of rollers which apply adhesive to the surface of the films not within the recesses.

The films then move past respective filling stations 76, 78 where metered doses of material are supplied to each outer shell half as it passes below the station. Suitable filling equipment for supplying metered doses of liquid materials (eg syringe pumps, peristaltic pumps etc) and for supplying metered doses of powdered or particulate materials are well known. Typical volume fills are in the range 0.1 to 3.0 mls per capsule half.

The filled outer shell halves then move inwardly with the vacuum bed, past guide rollers 64, 66 around which pass lengths of septum-forming films 54, 56. The septum-forming films adhere to the non-deformed parts of films 50 and 52 under the action of the previously applied adhesive, closing off the half capsules.

The thus formed half capsules move inwardly with the vacuum belt past further adhesive stations 80, 82 which act to apply adhesive to the top surface of the septum-forming films.

The capsule halves are brought together between adjacent sides of the vacuum belts and the two septums adhere together by adhesive action. At this point, the capsules are loosely stuck together.

The films with arrays of capsules therebetween are fed to a sealing station comprising two heater blocks 88, 90 mounted on pneumatic rams that reciprocate towards and away from each other in synchronism. The blocks act to heat and fully seal together the capsule halves, forming compartmented capsules in accordance with the invention. A knife edge (not shown) is provided on one of the blocks to cut the capsules from the remaining material. The cut capsules are collected below and the remaining film web material is passed to waste.

In a typical embodiment the films comprise HPMC having a thickness of about 120 nm. Such material is readily available commercially. For example, HPMC is available from Dow Chemicals (USA) and is made into a film by Cast Film Technologies (USA).

Optional coatings may be applied to the film material, eg upstream of the rollers. Different coatings may be applied to the different half-capsule forming films.

When treating HPMC, the films should be heated at heating stations to a temperature of about 85 to 90° C. so as to become thermoplastic and deformable.

A suitable adhesive for use with HPMC is HPMC with 60% propylene glycol, which can be applied warm or cold. Other possible adhesive/plasticizer materials include triacetin, monoacetin and ethyl lactate.

The adhesive formulation can also be applied before the forming heaters provided that it is of food grade and there is no reaction with the capsule contents. In such a case there will be a continuous coating of the adhesive present inside the formed capsule half. This can help with adhesion of the septum-forming film by causing a build up inside the seam.

For sealing HPMC, the heating block should be heated to a temperature in the range 150 to 170° C.

When using PVA instead of HPMC, heater temperatures must be much higher, about 150° C. to produce a thermoplastic film, with the heater block typically being heated to a temperature in the range 160 to 200° C.

The illustrated equipment can run at a rate capable of producing about 30,000 capsules per hour with a web width of about 600 mm.

A typical embodiment uses expanded HPMC for one capsule half and standard cast HPMC coated with sodium alginate for the other capsule half. The standard cast HPMC has a thickness of about 120 micron with a coating of alginate in the range 2 to 10 microns thick.

The application of the first adhesive is conveniently effected by rolling, extrusion or spraying, preferably by use of a roller, while application of the second adhesive is conveniently effected by a roller in contact with the film.

The capsules produced by the apparatus of FIG. 2 have a form generally corresponding to the capsule of FIG. 1, with septum 16 being constituted by two adhered together layers of film 54 and 56. The capsules include a short peripheral median flange (not shown in FIG. 1), aligned with and extending outwardly from the position of septum 16, constituted by portions of the four films 50, 52, 54, 56 adhered together to seal the compartments and capsule.

1. A delivery capsule having at least two separate chambers, the capsule including a dividing wall or septum defining in part two separate chambers, wherein the dividing wall or septum comprises two layers of material adhered together.

2-14. (canceled)

15. A delivery capsule having at least two separate chambers, the capsule including a dividing wall or septum defining in part two separate chambers, wherein the dividing wall or septum comprises two layers of material adhered together.

16. A capsule according to claim 15, wherein each chamber contains a different material.

18. A capsule according to claim 16, wherein each chamber contains a metered dose of a material

19. A capsule according to claim 15, wherein the dividing wall or septum comprises a median wall symmetrically arranged to form two chambers of similar size and shape.

20. A capsule according to claim 16, wherein the dividing wall or septum comprises a median wall symmetrically arranged to form two chambers of similar size and shape.

21. A capsule according to claim 17, wherein the dividing wall or septum comprises a median wall symmetrically arranged to form two chambers of similar size and shape.

22. A capsule according to claim 15, formed from a heat-sealable material that is capable of deforming plastically on heating and/or when partially solvated.

23. A capsule according to claim 16, formed from a heat-sealable material that is capable of deforming plastically on heating and/or when partially solvated

24. A capsule according to claim 17, formed from a heat-sealable material that is capable of deforming plastically on heating and/or when partially solvated

25. A capsule according to claim 19, formed from a heat-sealable material that is capable of deforming plastically on heating and/or when partially solvated

26. A capsule according to claim 22, wherein the capsule is formed from one or more materials selected from the group consisting of: hydroxy propyl methyl cellulose, pectin, polyethylene oxide, polyvinyl alcohol, alginate, polycaprolactone, and gelatinised starch based materials.

27. A capsule according to claim 26, wherein at least part of the capsule material carries a coating.

28. A capsule according to claim 15, wherein said at least two chambers are designed to release their contents under similar circumstances.

29. A capsule according to claim 16, wherein said at least two chambers are designed to release their contents under similar circumstances.

30. A capsule according to claim 17, wherein said at least two chambers are designed to release their contents under similar circumstances.

31. A capsule according to claim 19, wherein said at least two chambers are designed to release their contents under similar circumstances.

32. A capsule according to claim 22, wherein said at least two chambers are designed to release their contents under similar circumstances.

33. A capsule according to claim 26, wherein said at least two chambers are designed to release their contents under similar circumstances.

34. A capsule according to claim 27, wherein said at least two chambers are designed to release their contents under similar circumstances.

35. A capsule according to claim 15, wherein said at least two chambers are designed to release their contents under different circumstances.

36. A capsule according to claim 16, wherein said at least two chambers are designed to release their contents under different circumstances.

37. A capsule according to claim 17, wherein said at least two chambers are designed to release their contents under different circumstances.

38. A capsule according to claim 19, wherein said at least two chambers are designed to release their contents under different circumstances.

39. A capsule according to claim 22, wherein said at least two chambers are designed to release their contents under different circumstances.

40. A capsule according to claim 26, wherein said at least two chambers are designed to release their contents under different circumstances.

41. A capsule according to claim 27, wherein said at least two chambers are designed to release their contents under different circumstances.

42. A capsule according to claim 28, wherein said at least two chambers are designed to release their contents under different circumstances.

43. A capsule according to claim 35, wherein different chambers of the capsule are defined at least in part by different materials.

44. A capsule according to any one of claims 15, 16, 17, 19, 22, 26, 27, 28, or 35, wherein the capsule is formed at least in part from hydroxy propyl methyl cellulose.

45. A capsule according to claim 44, wherein at least part of the hydroxy propyl methyl cellulose is coated with alginate.

46. A method of encapsulation comprising supplying two films of material capable of deforming plastically on heating and/or when partially solvated; heating the films and/or applying solvent; forming the films into suitably shaped capsule portions; supplying respective substances to be encapsulated to capsule portions of each film; supplying a respective film of a dividing septum material to each of the filled capsule portions; and sealing the capsule portions and septum material together to form a capsule having at least two separate chambers.

47. Encapsulation apparatus comprising means for supplying two films of material to an encapsulation unit; means for plastically deforming each film to form suitably shaped capsule portions; means for supplying respective substances to be encapsulated to the respective capsule portions of each film; means for supplying a respective film of dividing septum material to each of the filled capsule portions; and means for sealing together the capsule portions and septum material to produce a capsule having at least two separate chambers.
